(19) United States
(12) Patent Application Publication
(10) Pub. No.: US 2009/0137661 A1
(43) Pub. Date: May 28, 2009

(54) PLANT DERIVED COMPOUNDS AND COMPOUND FORMULAE CONTAINING THE SAME FOR THE TREATMENT OF CERVICAL CANCER

(76) Inventors: Ming-Kuang Shih, Sinjhuang City (TW); Yu-Chou Chao, Taipei (TW); Ying-Chu Shih, Jhubei City (TW); Kuo-Kuei Huang, Jhudong Township (TW)

Correspondence Address: Muncy, Geissler, Olds & Lowe, PLLC, P.O. BOX 1364, FAIRFAX, VA, 22038-1364, US

(21) Appl. No.: 12/323,008

(22) Filed: Nov. 25, 2008

Related U.S. Application Data
(60) Provisional application No. 60/990,174, filed on Nov. 26, 2007.

(51) Int. Cl. A61K 31/352; A61K 31/19

(52) U.S. Cl. 514/453; 514/460; 514/568; 514/455

(57) ABSTRACT

A composition for reducing the activity of a cervical cancer cell is provided. The composition includes at least one of the following compounds: isopsoralen, triptolide, baicalein, gallic acid, quercetin, gossypol-acetic acid, baicalin, berberine hydrochloride, and derivatives thereof in a sufficient amount to reduce the activity of the cervical cancer cell in the subject.

### CROSS-REFERENCE TO RELATED PATENT APPLICATION

This application claims the benefit, pursuant to 35 U.S.C. §119(e), of U.S. provisional patent application No. 60/990,174 filed Nov. 26, 2007, which is incorporated herein by reference in its entirety.

### BACKGROUND

1. Field of Invention

The present invention relates to plant derived compounds for treating cervical cancer. More particularly, the present invention relates to compound formulae comprising said plant derived compounds for treating cervical cancer.

2. Description of Related Art

Cervical cancer is the second leading form of cancer among women in Taiwan, with more than 1000 women died from it each year. It has been proven that human papilloma virus (HPV) infection is a necessary factor in the development of nearly all cases of cervical cancer. In the U.S. and the EU, HPV vaccine effective against a few strains of HUMAN PAPILLOMA VIRUS that together are currently responsible for approximately 70% of all cervical cancers has been developed. In Taiwan, however, there is no significant advancement concerning cervical cancer accomplished.

Human papilloma viruses are non-enveloped, double-stranded DNA viruses that have icosahedral symmetry. The genome of human papilloma virus is surrounded by a capsid consisted of 72 capsomers. The HPV genome is frequently mutated in the host and approximately 250 HPV types have been identified wherein the structure of human papilloma virus varies depending on the territorial, living behavior, ethnic group, and infectious pathway. As previously mentioned, the HPV vaccine only covers some high-risk types; therefore, women should seek regular Pap smear screening, even after vaccination. Besides, the HPV vaccine should be given before infection occurs, therefore, HPV vaccines are targeted at girls and women before they begin having sex.

After the infection of human papilloma virus, the virus might trigger alterations in the cells of the cervix, which can lead to cervical cancer. Cervical cancer can be treated by carcinomectomy, radiation therapy, and/or chemotherapy.

In view of the foregoing, efforts are needed to provide treatment for cervical cancer.

### SUMMARY

In one aspect of the present invention, a composition for reducing the activity of a cervical cancer cell is provided. The composition comprises at least one of the following compounds: isopsoralen, triptolide, baicalein, gallic acid, quercetin, gossypol-acetic acid, baicalin, berberine hydrochloride, and derivatives thereof in a sufficient amount to reduce the activity of the cervical cancer cell in the subject.

In another aspect of the present invention, a composition for reducing the viral activity of a human papilloma virus in a virus-infected cell or a virus-infected subject is provided. The composition comprises at least one of the following compounds: baicalein, gallic acid, and derivatives thereof in a sufficient amount to reduce the viral activity in the virus-infected cell or the virus-infected subject.

In still another aspect of the present invention, a method of reducing the activity of a cervical cancer cell is provided. The method comprises administering to the cervical cancer cell a therapeutically effective amount of a composition comprising a compound selected from the group consisting of isopsoralen, triptolide, baicalein, gallic acid, quercetin, gossypol-acetic acid, baicalin, berberine hydrochloride, and derivatives thereof.

In yet another aspect of the present invention, a method of reducing the viral activity of a human papilloma virus in a virus-infected cell or a virus-infected subject is provided. The method comprises administering to the virus-infected cell or the virus-infected cell subject a therapeutically effective amount of a composition comprising at least one of the following compounds: baicalein, gallic acid, and derivatives thereof.

These and other features, aspects, and advantages of the present invention will become better understood with reference to the following description and appended claims. It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a line graph illustrating the relationship between the volume of the HPV 16 pseudovirus and the relative fluorescence unit thereof according to one experimental example of the present invention;

FIG. 2 is a line graph illustrating the relationship between the volume of the HPV 16 pseudovirus of FIG. 1 and the signal to background ratio thereof;

FIG. 3 is a line graph illustrating the relationship between the concentration of isopsoralen (Compound A) and the HeLa cell survival rate at 24, 48, and 72 hours after treatment according to experimental examples of the present invention;

FIG. 4 is a line graph illustrating the relationship between the concentration of triptolide (Compound B) and the HeLa cell survival rate at 24, 48, and 72 hours after treatment according to experimental examples of the present invention;

FIG. 5 is a line graph illustrating the relationship between the concentration of baicalein (Compound C) and the HeLa cell survival rate at 24, 48, and 72 hours after treatment according to experimental examples of the present invention;

FIG. 6 is a line graph illustrating the relationship between the concentration of gallic acid (Compound D) and the HeLa cell survival rate at 24, 48, and 72 hours after treatment according to experimental examples of the present invention;

FIG. 7 is a line graph illustrating the relationship between the concentration of quercetin (Compound E) and the HeLa cell survival rate at 24, 48, and 72 hours after treatment according to experimental examples of the present invention;

FIG. 8 is a line graph illustrating the relationship between the concentration of gossypol-acetic acid (Compound F) and the HeLa cell survival rate at 24, 48, and 72 hours after treatment according to experimental examples of the present invention;

FIG. 9 is a line graph illustrating the relationship between the concentration of baicalin (Compound G) and the HeLa cell survival rate at 24, 48, and 72 hours after treatment according to experimental examples of the present invention;

FIG. 10 is a line graph illustrating the relationship between the concentration of berberine hydrochloride (Compound H) and the HeLa cell survival rate at 24, 48, and 72 hours after treatment according to experimental examples of the present invention;

FIG. 11 is a line graph illustrating the relationship between the concentration of Doxorubin HCl (Control) and the HeLa cell survival rate at 24, 48, and 72 hours after treatment according to experimental examples of the present invention;

FIG. 12 is a line graph further illustrating the relationship between the concentration of triptolide (Compound B) and the HeLa cell survival rate at 24, 48, and 72 hours after treatment according to experimental examples of the present invention;

FIG. 13 is a graph illustrating the relationship between the concentration of baicalein (Compound C) and the HeLa cell survival rate (shown as line in the graph) and the relationship between the concentration of baicalein (Compound C) and HPV 16 pseudovirus infection rate (shown as blocks in the graph) at 48 hours after treatment according to experimental examples of the present invention;

FIG. 14 is a graph illustrating the relationship between the concentration of concentration of gallic acid (Compound D) and the HeLa cell survival rate (shown as line in the graph) and the relationship between the concentration of concentration of gallic acid (Compound D) and HPV 16 pseudovirus infection rate (shown as blocks in the graph) at 48 hours after treatment according to experimental examples of the present invention; and

FIG. 15 is a graph illustrating the relationship between the concentration of Carrageenan (Control) and the HeLa cell survival rate (shown as line in the graph) and the relationship between the concentration of Carrageenan (Control) and HPV 16 pseudovirus infection rate (shown as blocks in the graph) at 48 hours after treatment according to experimental examples of the present invention.

### DETAILED DESCRIPTION

Among those human papilloma viruses identified, 15 are classified as high-risk types (16, 18, 31, 33, 35, 39, 45, 51, 52, 56, 58, 59, 68, 73, and 82). Specifically, HPV 16 and HPV 18 are generally acknowledged to cause about 70% of cervical cancer cases. Albeit being denominated as “high-risk” types, the infection of high risk HPV will not necessarily cause cervical cancer. On the other hand, even the probable high-risk (26, 53, and 66) and the low-risk (6, 11, 40, 42, 43, 44, 54, 61, 70, 72, 81, and CP6108) types may cause cervical cancer.

Chinese herbal medicine has been practiced for thousands of years. Thanks to the advancement in modern science and medicine, it has been established that Chinese herbs are effective for preventing, treating, and/or ameliorating many ailments and illnesses. Also, Chinese herbs are known to induce fewer side effects in human body. Recently, Chinese herbal medicines compatible with or counteracting the side effects of conventional cancer therapies such as chemotherapy and radiation are proposed in both eastern and western countries.

To purse Chinese herbs capable of treating cervical cancer and/or human papilloma viruses, the inventor has investigated a variety of plants known to be Chinese herbs including Psoralea corylifolia L., Tripterygium wilfordii Hook. F., Scutellaria baicalensis Georgi, Cornus officinalis, Sophore flavescents Ait., Cotton seeds, and Coptis chinensis Franch among the others. The active compounds of said Chinese herbs were extracted and then purified by high performance liquid chromatography (HPLC) technique. The purity of each active compound was ≧97%. The plants and active compounds thereof and their structural formulae are listed in Table 1 below.

TABLE 1
Active Compounds of Selected Plants and Their Structural Formulae

| | | Active Compound/ | Molecular |
|---|---|---|---|
| | Plant Source | Structural Formula | Formula |
| A | Psoralea corylifolia L. | 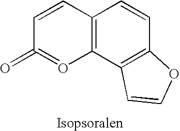 | C₁₁H₆O₃ |
| B | Tripterygium wilfordii Hook. F. | 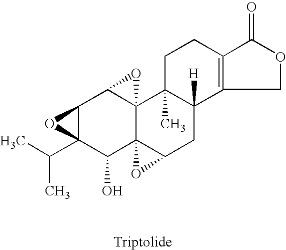 | C₂₀H₂₄O₆ |
| C | Scutellaria baicalensis Georgi | 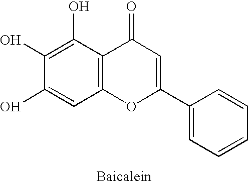 | C₁₅H₁₀O₅ |
| D | Cornus officinalis | 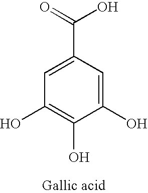 | (HO)₃C₆H₂CO₂H |
| E | Sophore flavescents Ait. | 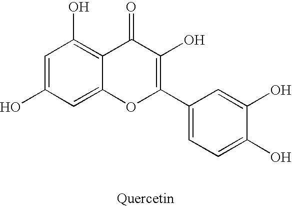 | C₁₅H₁₀O₇ |
| F | Cotton Seeds | 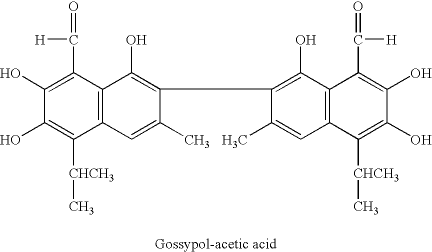 | C₃₀H₃₀O₈•C₂H₄O₂ |
| G | Scutellaria baicalensis Georgi | 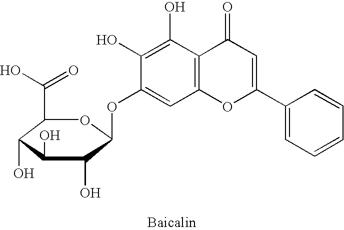 | C₂₁H₁₈O₁₁ |
| H | Coptis chinensis Franch | 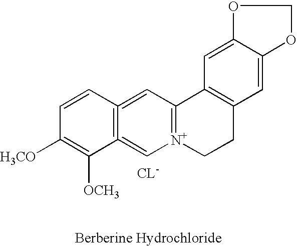 | C₂₀H₁₈NO₄•Cl |

According to one aspect of the present invention, in vitro studies have been performed to demonstrate the usefulness of compounds described herein for reducing the activity of a cervical cancer cells (HeLa cells).

According to another aspect of the present invention, in vitro studies have been performed to demonstrate the usefulness of compounds described herein for reducing the viral activity of a human papilloma virus in a virus-infected cell and/or a virus-infected subject.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. The use of examples anywhere in this specification including examples of any terms discussed herein is illustrative only, and in no way limits the scope and meaning of the invention or of any exemplified term. The invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, the term “treatment” covers any administration or application of remedies for disease in a human, and includes inhibiting the disease, arresting its development, or relieving the disease, for example, by causing regression, or restoring or repairing a lost, missing, or defective function; or stimulating an inefficient process. The term includes obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. Thus, the invention provides both treatment and prophylaxis. It includes (1) preventing the disorder from occurring or recurring in a subject who may be predisposed to the disorder but is not yet symptomatic, (2) inhibiting the disorder, such as arresting its development, (3) stopping or terminating the disorder or at least its associated symptoms, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as the cell activity of cancer cells or the virus-infected cells. For example, cell activity can be cell proliferating activity or cell metabolic activity.

As used herein, the term “therapeutically effective amount” refers to an amount which, when administered to a subject, achieves a desired effect on the subject. For example, an effective amount of the composition according to one embodiment of the present invention is an amount that reduces the activity of a cervical cancer cell of the subject. According to another embodiment of the present invention, an effective amount of the composition is an amount that reduces the viral activity of a human papilloma virus in a virus-infected cell or a virus-infected subject. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction, and the severity of the condition may be necessary, and will be ascertainable with routine experimentation by those skilled in the art.

As used herein, the term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosage and concentration employed and is compatible with other ingredients of the formulation.

### Materials and Methods

Biological Materials and Chemicals

HeLa cell line was obtained from Dr. S. S. Shen (Biomedical Engineering Research Laboratories of Industrial Technology Research Institute of Taiwan). HeLa cells are adherent cells and were propagated and maintained in Dulbecco's modified Eagle's medium (DMEM) supplemented with 10% fetal bovine serum (FBS), 1.5 g sodium bicarbonate (NaHCO₃), 1 mM sodium pyruvate, and 0.1 mM non-essential amino acid.

Human umbilical vein endothelial cells (HUVEC) were purchased from Food Industry Research and Development Institute in Hsin-Chu City, Taiwan. HUVEC cells were propagated and maintained in Medium 199 supplemented with 10% FBS, Heparin, and EGFP.

Human 293FT cell line was purchased from Invitrogen Corporation (California, USA). 293FT cells were propagated and maintained in DMEM supplemented with 10% FBS, 0.1 mM non-essential amino acid, and 500 ug/ml geneticin (G418).

Plasmids p16sheLL and pCIneoEGFP were obtained from John T, Schiller, Ph.D. of National Cancer Institute, USA.

DPBS-Mg Buffer was consisted of 100 ml of DPBS, 475 μl of 2M MgCl₂, and 1 ml of 100× antibiotic stock.

DMEM, Medium 199, geneticin, non-essential amino acid, lipofectamine 2000, cell culture reagent Opti-MEM-I, and DPBS were purchased from Gibco Invetrogen (N.Y., USA). FBS was purchased from Biological Industries Ltd. (Israel). Heparin, EGFP, Brij-58, 3-(4,5-cimethylthiazol-2-yl)-2,5-diphenyl tetrazolium bromide (MTT) were purchased from Sigma-Aldrich Corp. (USA).

Doxorubicin HCl, a commercially available chemotherapy drug used in cancer treatment, was purchased from Sigma-Aldrich Co.

Corning® 96-well plate, Cat:3603 (Black plate, Clear bottom with lid), was purchased from Corning Inc. (USA).

Cytotoxicity Assay (MTT Assay)

The cytotoxicity of the compounds set forth herein to HeLa cells and HUVEC cells were determined by the MTT assay. The HUVEC cells were used to understand the cytotoxicity of said compounds to normal human cells. Besides, doxorubicin HCl was used as a comparative example so as to investigate the efficacy of the compounds according to the embodiments of the present invention.

Briefly, the MTT assay comprises the following steps:
(A) Cells were seeded in 96-well plates 3 hours before treatment, wherein the density of HeLa cell is 1×10⁴ cells per well and the density of HUVEC cell is 2×10⁴ cells per well;
(B) Cells were then treated with various concentrations of tested samples and incubated for a predetermined period (24, 48, and 72 hours);
(C) After the predetermined periods expired, the tested samples were removed and 100 μl MTT reagent (1 mg/ml) was added to each well;
(D) The plates were incubated at 37° C. with 5% of CO₂ for 5 hours;
(E) The MTT reagent was removed and 100 μl DMSO was added to each well;
(F) The optical density (O.D.) of each well was determined by ELISA reader at a wavelength of 560 nm.

Each experiment was repeated for 3 times and the result shown in the tables herein were the mean value of the 3 repetitions. The O.D. of the vehicle control and experimental groups were recorded. The cell survival rate of each sample was calculated as follows:

Cell survival rate(%)=O.D._{control}/O.D.ₑₓₚₑᵣᵢₘₑₙₜₐₗ*100%

Then, the cell survival rate was plotted against the compound concentration and the IC₅₀ value of each compound was calculated by the GraFit data analysis software (Erithacus Software Ltd.).

Preparation of Green Fluorescent Protein (GFP) Expressing HPV 16 Pseudovirus

239 FT cells were co-transfected with codon-modified papillomavirus capsid genes, L1 and L2, plasmid: p16shell, and a GFP reporter plasmid, pClneo-GFP, to monitor the infectivity of the stock.

The GFP-expressing HPV 16 pseudoviruses were prepared and harvested in accordance with the protocol provided by the National Cancer Institute (USA). Said protocol is available on line at http://home.ccr.cancer.gov/lco/production.asp which is incorporated herein by reference in its entirety.

In the present application, the GFP-expressing HPV 16 pseudoviruses used have titers of about 4*10⁸ infectious units per ml.

Screening for Anti-Infection Compounds

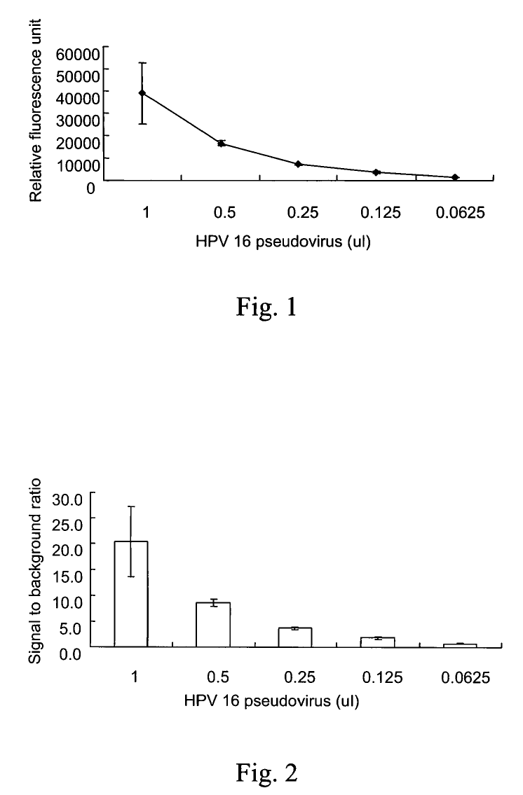

HeLa cells were seeded in 96-well plates at a density of 6×10³ cells per well. 24 hours later, 2×HPV 16 pseudoviruses of various volumes were added. 48 hours later, the fluorescence units of the samples were determined by an ELISA reader and the result was shown in FIG. 1. The signal to background ratio of each volume of the HPV 16 pseudoviruses was illustrated in FIG. 2.

When choosing screening platform, the infection rate should be around 20%. Hence, 0.25 μl of HPV 16 pseudovirus per well was used as the platform for screening anti-infection compounds. Under this dosage, the signal to background ratio is 2.0-2.5.

The screening was conducted as follows:
(A) HeLa cells were seeded in 96-well plates at a density of 6×10³ cells per well 24 hours before treatment;
(B) Cells were then treated with various concentrations of tested samples, infected with 0.25 μl of HPV 16 pseudovirus per well, and incubated at 37° C. for 48 hours;
(C) The fluorescence units (Excitation: 485 nm, Emission: 535 nm) of the samples were detected by an ELISA reader, and MTT assay according to the preceding description was performed.

Carrageenan was used as positive control. Each experiment was repeated for 2 times and the result shown in the tables herein were the mean value of the 2 repetitions. The cell survival rate and IC₅₀ of each sample were obtained in accordance with the method stated above.

### Results

Cytotoxicity of Active Compounds A-H to HeLa Cells

According to one aspect of the present invention, a series of experiments were conducted to determine the cytotoxicity of each compound to HeLa cells. The active compounds listed in table 1 and a positive control compound, Doxorubin HCl, of various concentrations were given to HeLa cells and HUVEC cells. In these experiments, HeLa cells not treating with active compounds A-H and Doxorubin HCl (addition concentration=0 μg/ml) were used as negative controls. The survival rates of HeLa cells and HUVEC cells at 24, 48, and 72 hours after treatment were determined in accordance with the methods set forth in the Materials and Methods section. The results of MTT assays are shown in tables 2 and 3. As used herein, compounds A-H stand for isopsoralen, triptolide, baicalein, gallic acid, quercetin, gossypol-acetic acid, baicalin, and berberine hydrochloride, respectively.

TABLE 2
Active Compounds of various Concentration and HeLa Cell Survival
Rate at Predetermined Times
Compound A: Isopsoralen

| | 0 μg/ml | 0.125 μg/ml | 1.25 μg/ml | 12.5 μg/ml | 125 μg/ml |
|---|---|---|---|---|---|
| 24 hrs | 100 | 91 | 88 | 70 | 18 |
| 48 hrs | 100 | 99 | 75 | 30 | 11 |
| 72 hrs | 100 | 98 | 69 | 10 | 7 |

Compound B: Triptolide

| | 0 μg/ml | 0.001 μg/ml | 0.01 μg/ml | 0.1 μg/ml | 1 μg/ml |
|---|---|---|---|---|---|
| 24 hrs | 100 | 98 | 89 | 33 | 26 |
| 48 hrs | 100 | 94 | 37 | 14 | 12 |
| 72 hrs | 100 | 81 | 15 | 9 | 9 |

Compound C: Baicalein

| | 0 μg/ml | 0.125 μg/ml | 1.25 μg/ml | 12.5 μg/ml | 125 μg/ml |
|---|---|---|---|---|---|
| 24 hrs | 100 | 104 | 126 | 109 | 134 |
| 48 hrs | 100 | 107 | 110 | 73 | 68 |
| 72 hrs | 100 | 99 | 106 | 66 | 29 |

Compound D: Gallic acid

| | 0 μg/ml | 0.125 μg/ml | 1.25 μg/ml | 12.5 μg/ml | 125 μg/ml |
|---|---|---|---|---|---|
| 24 hrs | 100 | 85 | 84 | 90 | 17 |
| 48 hrs | 100 | 96 | 91 | 82 | 11 |
| 72 hrs | 100 | 101 | 103 | 99 | 8 |

Compound E: Quercetin

| | 0 μg/ml | 0.1 μg/ml | 1 μg/ml | 10 μg/ml | 100 μg/ml |
|---|---|---|---|---|---|
| 24 hrs | 100 | 93 | 96 | 83 | — |
| 48 hrs | 100 | 102 | 102 | 51 | 32 |
| 72 hrs | 100 | 95 | 91 | 33 | 12 |

Compound F: Gossypol-acetic acid

| | 0 μg/ml | 0.1 μg/ml | 1 μg/ml | 10 μg/ml | 100 μg/ml |
|---|---|---|---|---|---|
| 24 hrs | 100 | 101 | 97 | 80 | 17 |
| 48 hrs | 100 | 103 | 105 | 52 | 13 |
| 72 hrs | 100 | 98 | 98 | 40 | 10 |

Compound G: Baicalin

| | 0 μg/ml | 0.1 μg/ml | 1 μg/ml | 10 μg/ml | 100 μg/ml |
|---|---|---|---|---|---|
| 24 hrs | 100 | 104 | 103 | 109 | 68 |
| 48 hrs | 100 | 94 | 96 | 98 | 50 |
| 72 hrs | 100 | 97 | 97 | 97 | 37 |

Compound H: Berberine hydrochloride

| | 0 μg/ml | 0.1 μg/ml | 1 μg/ml | 10 μg/ml | 100 μg/ml |
|---|---|---|---|---|---|
| 24 hrs | 100 | 89 | 87 | 82 | 28 |
| 48 hrs | 100 | 98 | 84 | 71 | 17 |
| 72 hrs | 100 | 98 | 91 | 62 | 10 |

Control: Doxorubin HCl

| | 0 | | | | |
|---|---|---|---|---|---|
| | μg/ml | 0.00058 μg/ml | 0.0058 μg/ml | 0.058 μg/ml | 0.58 μg/ml |
| 24 hrs | 100 | 96 | 95 | 84 | 46 |
| 48 hrs | 100 | 100 | 92 | 81 | 23 |
| 72 hrs | 100 | 97 | 89 | 72 | 11 |

It can be seen in table 2, at each predetermined time, the cell survival rates of HeLa cell samples treated with active compounds A-H were lower than that of the negative controls. As comparing with Doxorubicin HCl, active compounds such as isopsoralen (compound A), triptolide (compound B), gallic acid (compound D), gossypol-acetic acid (compound F), and berberine hydrochloride (compound H) exhibited higher cytotoxicity to HeLa cells under the corresponding conditions. For example, when treated with 100 μg/ml of triptolide, the HeLa cell survival rates at 24, 48, and 72 hours are 20%, 13%, and 9%, respectively. On the other hand, the HeLa cells treated with 0.58 μg/ml of Doxorubicin HCl have a cell survival rate of 46%, 23%, and 11% at 24, 48, and 72 hours, respectively.

TABLE 3
Active Compounds of various Concentration and HUVEC Cell Survival
Rate at Predetermined Times
Compound A: Isopsoralen

| | 0 μg/ml | 0.125 μg/ml | 1.25 μg/ml | 12.5 μg/ml | 125 μg/ml |
|---|---|---|---|---|---|
| 24 hrs | 100 | 103 | 103 | 100 | 67 |
| 48 hrs | 100 | 104 | 96 | 83 | 31 |
| 72 hrs | 100 | 92 | 87 | 80 | 19 |

Compound B: Triptolide

| | 0 μg/ml | 0.001 μg/ml | 0.01 μg/ml | 0.1 μg/ml | 1 μg/ml |
|---|---|---|---|---|---|
| 24 hrs | 100 | 93 | 94 | 59 | 71 |
| 48 hrs | 100 | 102 | 111 | 36 | 35 |
| 72 hrs | 100 | 108 | 114 | 23 | 23 |

Compound C: Baicalein

| | 0 μg/ml | — | 1.25 μg/ml | 12.5 μg/ml | 125 μg/ml |
|---|---|---|---|---|---|
| 24 hrs | 100 | — | 109 | 88 | 199 |
| 48 hrs | 100 | — | 94 | 75 | 78 |
| 72 hrs | 100 | — | 83 | 69 | 57 |

Compound E: Quercetin

| | 0 μg/ml | 0.1 μg/ml | 1 μg/ml | 10 μg/ml | 100 μg/ml |
|---|---|---|---|---|---|
| 24 hrs | 100 | 104 | 108 | 101 | 120 |
| 48 hrs | 100 | 102 | 95 | 87 | 51 |
| 72 hrs | 100 | 98 | 94 | 80 | 26 |

Compound F: Gossypol-acetic acid

| | 0 μg/ml | 0.1 μg/ml | 1 μg/ml | 10 μg/ml | 100 μg/ml |
|---|---|---|---|---|---|
| 24 hrs | 100 | 93 | 88 | 80 | 20 |
| 48 hrs | 100 | 104 | 104 | 85 | 17 |
| 72 hrs | 100 | 103 | 98 | 71 | 17 |

Compound G: Baicalin

| | 0 μg/ml | — | 1.25 μg/ml | 12.5 μg/ml | 125 μg/ml |
|---|---|---|---|---|---|
| 24 hrs | 100 | — | 98 | 97 | 81 |
| 48 hrs | 100 | — | 96 | 101 | 76 |
| 72 hrs | 100 | — | 88 | 75 | 56 |

Compound H: Berberine hydrochloride

| | 0 μg/ml | — | 1 μg/ml | 10 μg/ml | 100 μg/ml |
|---|---|---|---|---|---|
| 24 hrs | 100 | — | 89 | 76 | 23 |
| 48 hrs | 100 | — | 81 | 64 | 16 |
| 72 hrs | 100 | — | 77 | 51 | 15 |

Control: Doxorubin HCl

| | 0 μg/ml | — | 0.145 μg/ml | 0.290 μg/ml | 0.580 μg/ml |
|---|---|---|---|---|---|
| 24 hrs | 100 | — | 111 | 100 | 90 |
| 48 hrs | 100 | — | 77 | 56 | 19 |
| 72 hrs | 100 | — | 68 | 38 | 14 |

It can be seen in tables 2 and 3, under specific concentrations, many of the above-mentioned active compounds possess higher selectivity to HeLa cells over HUVEC cells and thus is more suitable to be used in a composition for reducing the activity of a cervical cancer cell in a subject. Take triptolide (Compound B) for example, at 72 hours after treatment, HUVEC cells and HeLa cells treated with 0.01 μg/ml of triptolide have cell survival rates of 114% and 15%, respectively.

IC₅₀ of Active Compounds A-H for Inhibiting HeLa Cells

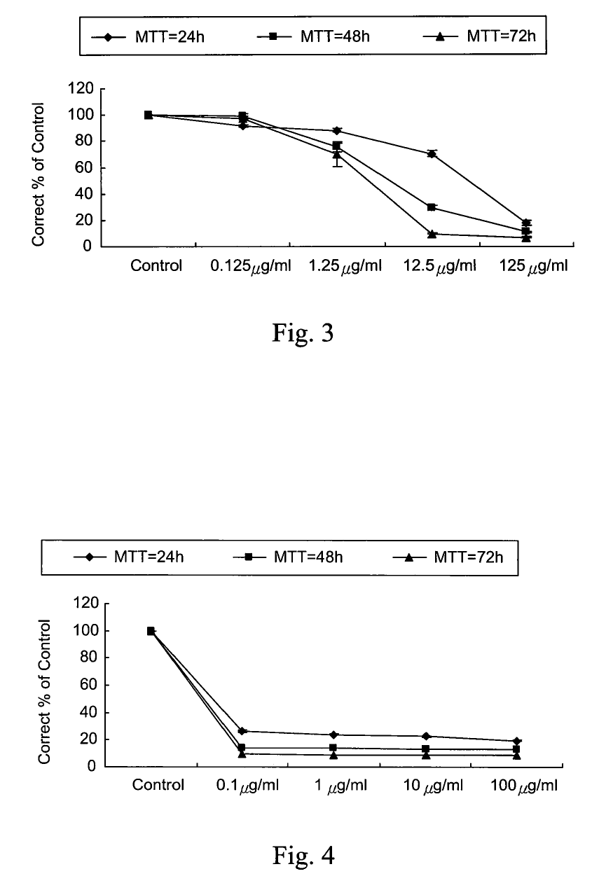

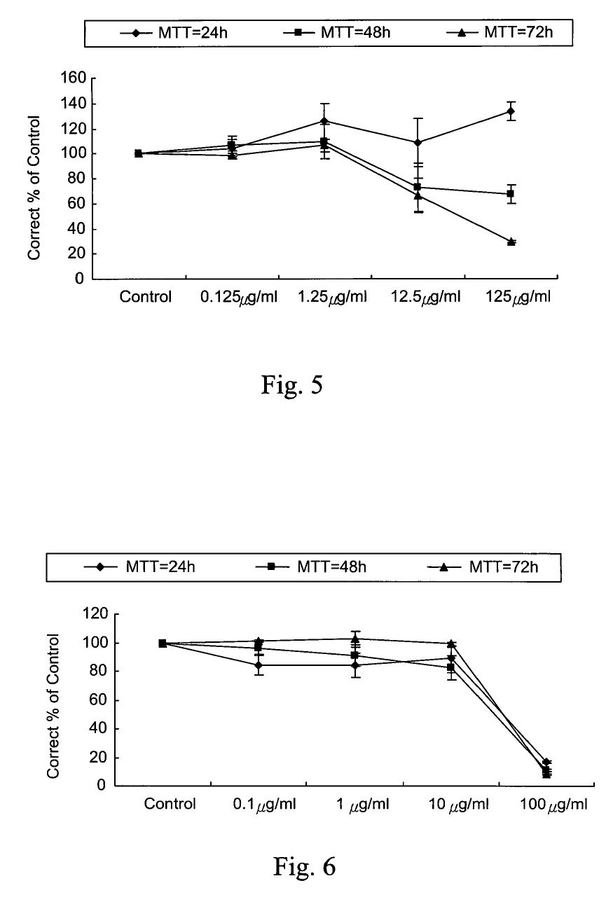

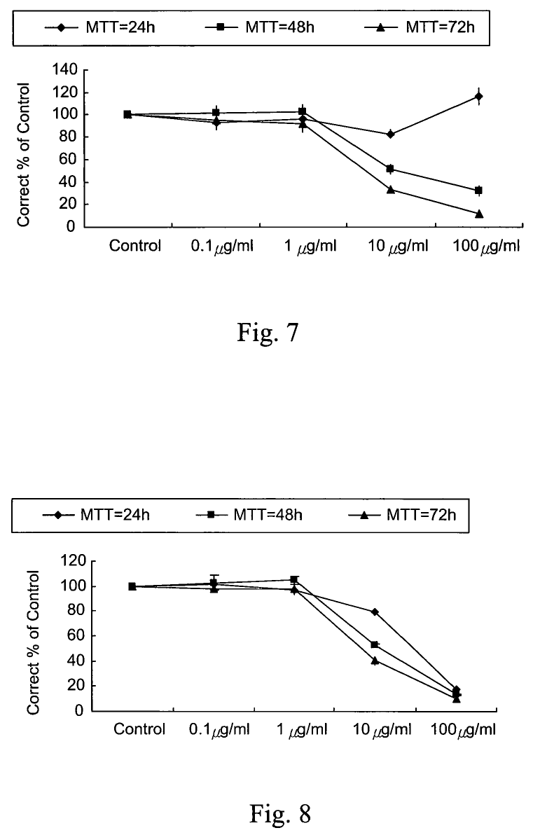

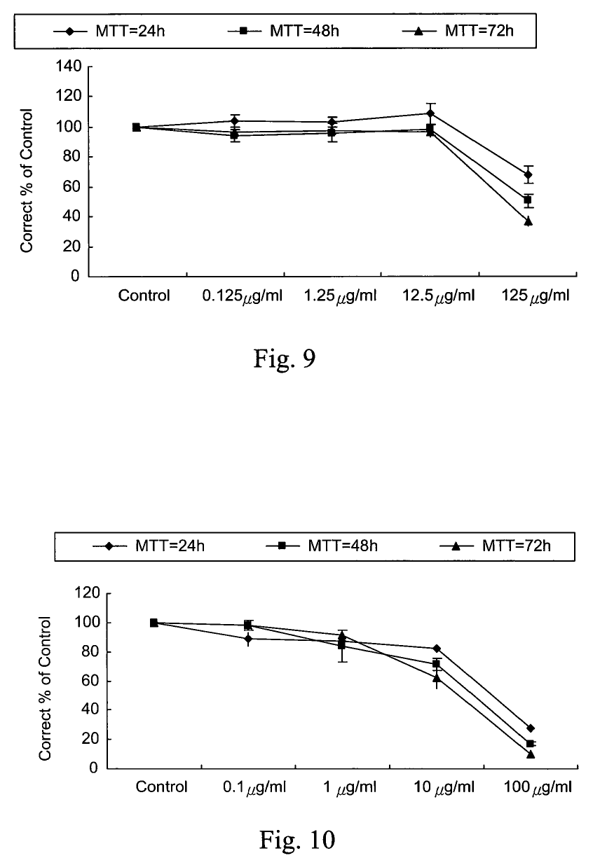

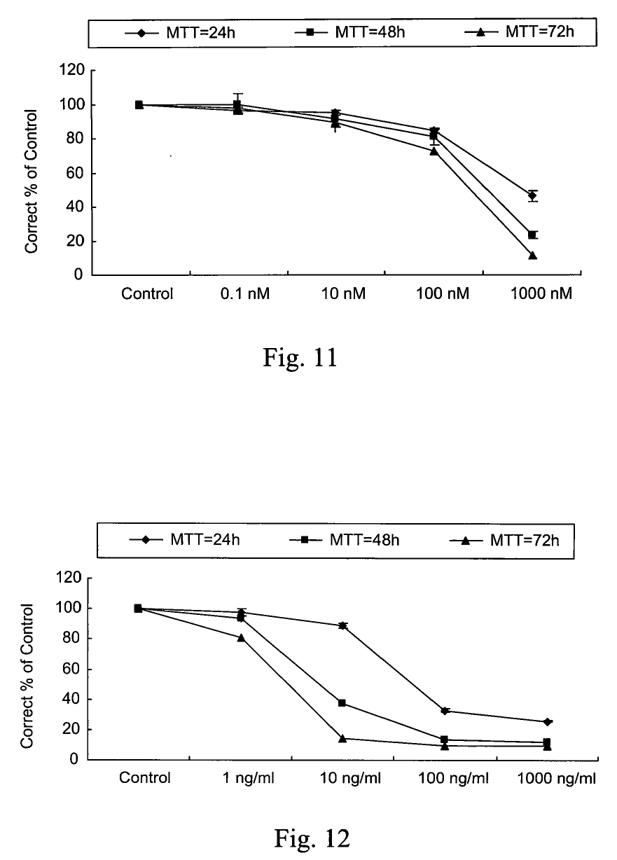

Line graphs were used to plot data recorded in both table 2 and table 3 so as to illustrate the relationship between compound concentrations and cell survival rates. FIGS. 3-11 are line graphs wherein HeLa cell survival rate of each experiment and control were plotted against the compound concentration used. The IC₅₀ value of each compound for inhibiting HeLa and HUVEC cells at a predetermined time was calculated by the GraFit data analysis software and the results were listed in table 4. The ratio of the IC₅₀ between HUVEC cells and HeLa cells was calculated for each compound and the results were also listed in table 4.

TABLE 4
IC₅₀ of Active Compounds and Control Compound for Inhibiting
HeLa Cells and HUVEC Cells at Predetermined Times
Compound A: Isopsoralen

| | IC₅₀ at 24 hours | IC₅₀ at 48 hours | IC₅₀ at 72 hours |
|---|---|---|---|
| | (μg/ml) | (μg/ml) | (μg/ml) |
| HeLa | 27 | 5 | 2 |
| HUVEC | >125 | 57 | 38 |
| HUVEC/HeLa | >4.6 | 11.4 | 19 |
| ratio | | | |

Compound B: Triptolide

| | IC₅₀ at 24 hours | IC₅₀ at 48 hours | IC₅₀ at 72 hours |
|---|---|---|---|
| | (ng/ml) | (ng/ml) | (ng/ml) |
| HeLa | 72.08 | 7.24 | 2.87 |
| HUVEC | >1000 | 151 | 84 |
| HUVEC/HeLa | >13.9 | 20.9 | 29.3 |
| ratio | | | |
| | IC₅₀ at 24 hours | IC₅₀ at 48 hours | IC₅₀ at 72 hours |
| | (μg/ml) | (μg/ml) | (μg/ml) |

Compound C: Baicalein

| | | | |
|---|---|---|---|
| HeLa | >125 | >125 | 37 |
| HUVEC | >125 | >125 | >125 |
| HUVEC/HeLa | — | — | >3.4 |
| ratio | | | |

Compound D: Gallic acid

| | | | |
|---|---|---|---|
| HeLa | 37 | 26 | 45 |

Compound E: Quercetin

| | | | |
|---|---|---|---|
| HeLa | >100 | 20 | 6 |
| HUVEC | >100 | 104 | 36 |
| HUVEC/HeLa | — | 5.2 | 6 |
| ratio | | | |

Compound F: Gossypol-acetic acid

| | | | |
|---|---|---|---|
| HeLa | 29 | 12 | 8 |
| HUVEC | 30 | 33 | 23 |
| HUVEC/HeLa | 1.0 | 2.8 | 2.9 |
| ratio | | | |

Compound G: Baicalin

| | | | |
|---|---|---|---|
| HeLa | >125 | >125 | 92 |
| HUVEC | >125 | >125 | 218 |
| HUVEC/HeLa | — | — | 2.4 |
| ratio | | | |

Compound H: Berberine hydrochloride

| | | | |
|---|---|---|---|
| HeLa | 39 | 21 | 15 |
| HUVEC | 30 | 15 | 9 |
| HUVEC/HeLa | 0.8 | 0.7 | 0.6 |
| ratio | | | |

Control: Doxorubin HCl

| | | | |
|---|---|---|---|
| HeLa | 0.48 | 0.20 | 0.12 |
| HUVEC | >0.58 | 0.299 | 0.22 |
| HUVEC/HeLa | >1.2 | 1.5 | 1.8 |
| ratio | | | |

As can be seen in table 4, all of the active compounds A-H can effectively reduce the activity of HeLa cells. Hence, according to one aspect of the present invention, a composition for reducing the activity of a cervical cancer cell comprises at least one of the following compounds: isopsoralen, triptolide, baicalein, gallic acid, quercetin, gossypol-acetic acid, baicalin, berberine hydrochloride, and derivatives thereof in a sufficient amount to reduce the activity of the cervical cancer cell in the subject.

As those skilled in the art could appreciate, the higher ratio of the IC₅₀ between HUVEC cells and HeLa cells suggests that the compound might have higher selectivity to HeLa cells over HUVEC cells and thus is more suitable to be used in a composition for reducing the activity of a cervical cancer cell in a subject. Therefore, according to one embodiment of the present invention, a composition for reducing the activity of a cervical cancer cell comprises at least one of the following compounds: isopsoralen, triptolide, baicalein, quercetin, gossypol-acetic acid, baicalin, and derivatives thereof in a sufficient amount to reduce the activity of the cervical cancer cell in the subject.

Compound Formulae

According to another embodiment of the present invention, compound formulae exhibiting synergistic effect were also provided. Compound formulae containing two or more active compounds were also tested for the cytotoxicity to HeLa cells and some selected results were shown in table 5.

TABLE 5
IC₅₀ of Compound Formulae for Inhibiting HeLa Cells at
48 Hours after Treatment

| Compound Formula | Composition | IC₅₀ μg/ml |
|---|---|---|
| M1 | Compound H:Compound A = 2:1 | 0.14 |
| M2 | Compound C:Compound A = 2:1 | 0.3 |
| M3 | Compound G:Compound A = 2:1 | 0.2 |
| M4 | Compound H:Compound E = 2:1 | 0.19 |
| M5 | Compound C:Compound E = 2:1 | 0.72 |
| M6 | Compound H:Compound B = 3:1 | 0.62 (ng/ml) |
| M7 | Compound C:Compound B = 3:1 | 1.28 (ng/ml) |
| M8 | Compound G:Compound B = 3:1 | 0.77 (ng/ml) |
| M9 | Compound H:Compound F = 2:1 | 0.08 |
| M10 | Compound C:Compound F = 2:1 | 0.1 |
| M11 | Compound G:Compound F = 2:1 | 0.13 |

In tables 4 and 5, it can be seen that the IC₅₀ values of the compound formulae were much smaller than the IC₅₀ values of the respective component compounds alone and thus exhibited significant synergistic effect. Take compound formula M1 (IC₅₀=0.14) as an example, compound formula M1 comprised compound H (berberine hydrochloride, IC₅₀=21) and compound A (isopsoralen, IC₅₀=11.4) and IC₅₀ of the compound formula M1 was 0.14, which is significantly lower than the component compounds.

Hence, one example of the present invention provides a compound formula comprising at least one compound of baicalein, baicalin, berberine hydrochloride, and derivatives thereof, and at least one compound of isopsoralen, triptolide, quercetin, gossypol-acetic acid, and derivatives thereof.

Screening for Anti-Infection Compounds

According to another aspect of the present invention, a series of experiments were conducted to determine if the active compounds A-H were capable of inhibiting HPV 16 pseudoviruses from infecting HeLa cells. The active compounds listed in table 1 and a positive control compound, carrageenan, of various concentrations were added into the screening platform mentioned above.

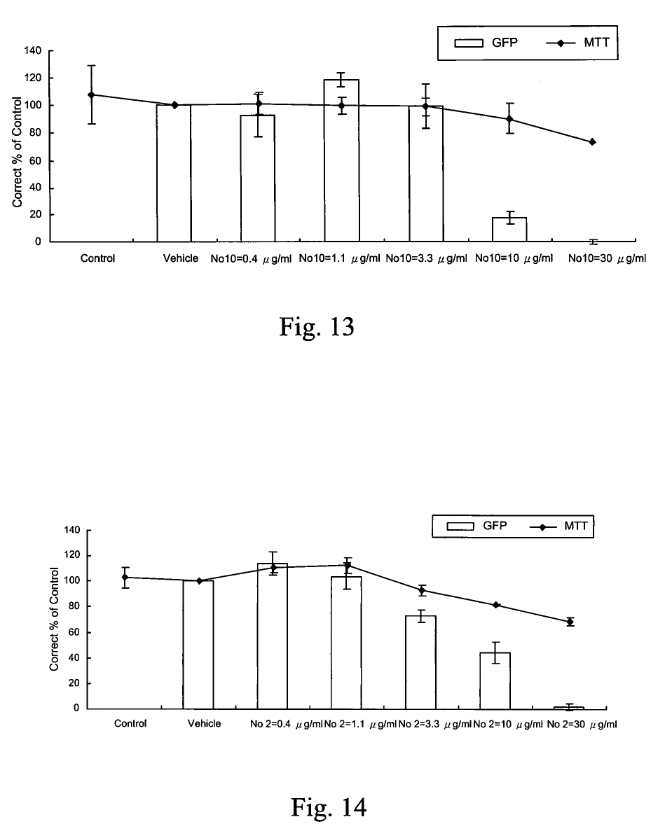

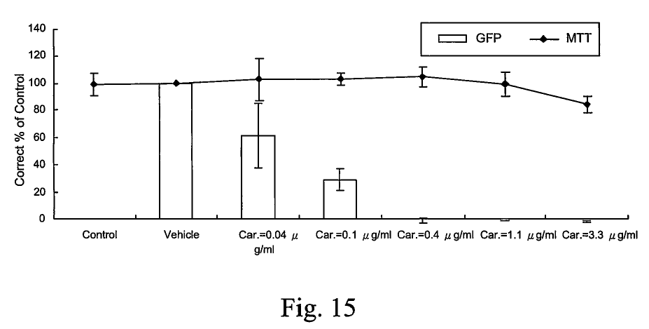

Selected test results were shown in FIGS. 13-15, wherein blocks indicated the amount of HPV 16 pseudoviruses in the samples and the results of the MTT assay were plotted as line graph. FIG. 15 demonstrates the test results of carrageenan. The IC₅₀ of carrageenan for inhibiting HPV 16 pseudoviruses is about 0.07 μg/ml, which is in agreement with reports from literature (See, for example, Carrageenan Is a Potent Inhibitor of Papillomavirus Infection, PloS Pathogens. 2006; 2:617).

According to the test results, it was found that under suitable concentrations, baicalein (compound C) and gallic acid (compound D) could inhibit HPV 16 pseudoviruses from infecting HeLa cells while not possessing significant cytotoxicity to HeLa cells. From the data presented in FIGS. 13 and 14, it was calculated that the IC₅₀ values of baicalein and gallic acid for inhibiting HPV 16 pseudoviruses were 8.2 and 8.9 μg/ml, respectively. Meanwhile, the survival rates of HeLa cells treated by baicalein and gallic acid were higher than about 80%.

### EMBODIMENTS

From the results set forth in this specification and other experiments conducted by the inventor, the following embodiments of the present invention are provided.

In one aspect of the present invention, a composition for reducing the activity of a cervical cancer cell in a subject is provided. The composition comprises at least one of the following compounds: isopsoralen, triptolide, baicalein, gallic acid, quercetin, gossypol-acetic acid, baicalin, berberine hydrochloride, and derivatives thereof in a sufficient amount to reduce the activity of the cervical cancer cell in the subject. According to the principles and spirits of the present invention, the subject to be treated can be a human having cervical cancer.

According to one embodiment of the present invention, the composition can be a simple formula that contains only one active compound set forth herein.

According to another embodiment of the present invention, the composition can be a compound formula that contains at least two active compounds set forth herein. In these compound formulae, the active compounds in the composition can exhibit synergistic effect. For example, the compound formula may comprise at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

According to yet another embodiment of the present invention, the composition further comprises a pharmaceutically acceptable carrier. Suitable pharmaceutically acceptable carriers include, but are not limited to, water, dextrose, glycerol, saline, ethanol, and combinations thereof. The pharmaceutically acceptable carrier can contain additional agents such as wetting or emulsifying agents, pH buffering agents, or adjuvants which enhance the effectiveness of the formulation. Other materials such as anti-oxidants, humectants, viscosity stabilizers, and similar agents can be added as necessary.

In another aspect of the present invention, a composition for reducing the viral activity of a human papilloma virus in a virus-infected cell or a virus-infected subject is provided. The composition comprises at least one of the following compounds: baicalein, gallic acid, and derivatives thereof in a sufficient amount to reduce the viral activity in the virus-infected cell or the virus-infected subject. According to the principles and spirits of the present invention, the subject to be treated can be a human having cervical cancer.

According to one embodiment of the present invention, the human papilloma virus to be treated is a high-risk human papilloma virus. For example, the high-risk human papilloma virus can be human papilloma virus 16 or human papilloma virus 18.

According to one embodiment of the present invention, the composition can be a simple formula that contains only one active compound set forth herein.

According to another embodiment of the present invention, the composition can be a compound formula that contains at least two active compounds set forth herein. In these compound formulae, the active compounds in the composition can exhibit synergistic effect. For example, the compound formula may comprise at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

According to yet another embodiment of the present invention, the composition further comprises a pharmaceutically acceptable carrier. Suitable pharmaceutically acceptable carriers can be those described above.

In still another aspect of the present invention, a method of reducing the activity of a cervical cancer cell is provided. The method comprises administering to the cervical cancer cell a therapeutically effective amount of a composition comprising a compound selected from the group consisting of isopsoralen, triptolide, baicalein, gallic acid, quercetin, gossypol-acetic acid, baicalin, berberine hydrochloride, and derivatives thereof. According to the principles and spirits of the present invention, the subject to be treated can be a human having cervical cancer.

According to one embodiment of the present invention, the composition to be administered can be a simple formula that contains only one active compound set forth herein.

According to another embodiment of the present invention, the composition to be administered can be a compound formula that contains at least two active compounds set forth herein. In these compound formulae, the active compounds in the composition can exhibit synergistic effect. For example, the compound formula may comprise comprises at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

According to yet another embodiment of the present invention, the composition further comprises a pharmaceutically acceptable carrier. Suitable pharmaceutically acceptable carriers can be those described above.

In pharmaceutical dosage forms, the compositions of the present invention can be administered in the form of their pharmaceutically acceptable salts, or they can also be used alone or in appropriate association, as well as in combination, with other pharmaceutically active compounds. The subject compositions are formulated in accordance to the mode of potential administration.

In yet another aspect of the present invention, a method of reducing the viral activity of a human papilloma virus in a virus-infected cell or a virus-infected subject is provided. The method comprises administering to the virus-infected cell or the virus-infected cell subject a therapeutically effective amount of a composition comprising at least one of the following compounds: baicalein, gallic acid, and derivatives thereof. According to the principles and spirits of the present invention, the subject to be treated can be a human having cervical cancer.

According to one embodiment of the present invention, the human papilloma virus to be treated is a high-risk human papilloma virus. For example, the high-risk human papilloma virus can be human papilloma virus 16 or human papilloma virus 18.

According to one embodiment of the present invention, the composition to be administered can be a simple formula that contains only one active compound set forth herein.

According to another embodiment of the present invention, the composition to be administered can be a compound formula that contains at least two active compounds set forth herein. In these compound formulae, the active compounds in the composition can exhibit synergistic effect. For example, the compound formula may comprise at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

Similarly, in pharmaceutical dosage forms, the compositions of the present invention can be administered in the form of their pharmaceutically acceptable salts, or they can also be used alone or in appropriate association, as well as in combination, with other pharmaceutically active compounds. The subject compositions are formulated in accordance to the mode of potential administration.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A composition for reducing the activity of a cervical cancer cell in a subject, comprising a compound selected from the group consisting of isopsoralen, triptolide, baicalein, gallic acid, quercetin, gossypol-acetic acid, baicalin, berberine hydrochloride, and derivatives thereof in a sufficient amount to reduce the activity of the cervical cancer cell in the subject.

2. The composition for reducing the activity of a cervical cancer cell of claim 1, wherein the composition is a compound formula.

3. The composition for reducing the activity of a cervical cancer cell of claim 2, wherein the compound formula comprises at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

4. The composition for reducing the activity of a cervical cancer cell of claim 1, wherein the subject is a human.

5. The composition for reducing the activity of a cervical cancer cell of claim 1, further comprising a pharmaceutically acceptable carrier.

6. A composition for reducing the viral activity of a human papilloma virus in a virus-infected cell or a virus-infected subject, comprising a compound selected from the group consisting of baicalein, gallic acid, and derivatives thereof in a sufficient amount to treat and reduce the viral activity in the virus-infected cell or the virus-infected subject.

7. The composition for reducing the viral activity of a human papilloma virus in a virus-infected cell or a virus-infected subject of claim 6, wherein the human papilloma virus is a high-risk human papilloma virus.

8. The composition for reducing the viral activity of a human papilloma virus in a virus-infected cell or a virus-infected subject of claim 7, wherein the high-risk human papilloma virus is human papilloma virus 16.

9. The composition for reducing the viral activity of a human papilloma virus in a virus-infected cell or a virus-infected subject of claim 7, wherein the high-risk human papilloma virus is human papilloma virus 18.

10. The composition for reducing the viral activity of a human papilloma virus in a virus-infected cell or a virus-infected subject of claim 6, wherein the composition is a compound formula.

11. The composition for reducing the viral activity of a human papilloma virus in a virus-infected cell or a virus-infected subject of claim 10, wherein the compound formula comprises at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

12. The composition for reducing the viral activity of a human papilloma virus in a virus-infected cell or a virus-infected subject of claim 6, wherein the subject is a human.

13. The composition for reducing the viral activity of a human papilloma virus in a virus-infected cell or a virus-infected subject of claim 6, further comprising a pharmaceutically acceptable carrier.

14. A method of reducing the activity of a cervical cancer cell, comprising administering to the cervical cancer cell a therapeutically effective amount of a composition comprising a compound selected from the group consisting of isopsoralen, triptolide, baicalein, gallic acid, quercetin, gossypol-acetic acid, baicalin, berberine hydrochloride, and derivatives thereof.

15. The method of reducing the activity of a cervical cancer cell of claim 14, wherein the composition is a compound formula.

16. The method of reducing the activity of a cervical cancer cell of claim 15, wherein the compound formula comprises at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

17. The method of reducing the activity of a cervical cancer cell of claim 14, wherein the subject is a human.

18. The method of reducing the activity of a cervical cancer cell of claim 14, wherein the composition further comprising a pharmaceutically acceptable carrier.

19. A method of reducing the viral activity of a human papilloma virus in a virus-infected cell or a virus-infected subject, comprising administering to the virus-infected cell or the virus-infected cell subject a therapeutically effective amount of a composition comprising a compound selected from the group consisting of baicalein, gallic acid, and derivatives thereof.

20. The method of reducing the viral activity of a human papilloma virus in a virus-infected cell or a virus-infected subject of claim 19, wherein the human papilloma virus is a high-risk human papilloma virus.

21. The method of reducing the viral activity of a human papilloma virus in a virus-infected cell or a virus-infected subject of claim 20, wherein the high-risk human papilloma virus is human papilloma virus 16.

22. The method of reducing the viral activity of a human papilloma virus in a virus-infected cell or a virus-infected subject of claim 20, wherein the high-risk human papilloma virus is human papilloma virus 18.

23. The method of reducing the viral activity of a human papilloma virus in a virus-infected cell or a virus-infected subject of claim 19, wherein the composition is a compound formula.

24. The method of reducing the viral activity of a human papilloma virus in a virus-infected cell or a virus-infected subject of claim 23, wherein the compound formula comprises at least one compound selected from the group consisting of baicalein, baicalin, berberine hydrochloride, and derivatives thereof, and at least one compound selected from the group consisting of isopsoralen, triptolide, quercetin, gossypol-acetic acid, and derivatives thereof.

25. The method of reducing the viral activity of a human papilloma virus in a virus-infected cell or a virus-infected subject of claim 19, wherein the subject is a human.

26. The method of reducing the viral activity of a human papilloma virus in a virus-infected cell or a virus-infected subject of claim 19, wherein the composition further comprising a pharmaceutically acceptable carrier.
